(19) United States
(12) Patent Application Publication
(10) Pub. No.: US 2013/0000649 A1
(43) Pub. Date: Jan. 3, 2013

(54) TRACHEAL TUBE WITH CONTROLLED-PROFILE CUFF

(75) Inventor: Sarah Hayman, Boulder, CO (US)

(73) Assignee: Nellcor Puritan Bennett LLC, Boulder, CO (US)

(21) Appl. No.: 13/171,760

(22) Filed: Jun. 29, 2011

(51) Int. Cl. A61M 16/04; B23P 11/00

(52) U.S. Cl. 128/207.15; 29/428

(57) ABSTRACT

The present disclosure describes systems and methods that utilize a tracheal tube with a reduced profile. An inflatable balloon cuff may be positioned within a recessed portion of an outer wall of the tracheal tube to reduce the overall profile of the tracheal tube. In this manner, protrusion of the inflatable balloon cuff from the tube is minimized, which protects the cuff from damage (e.g., snagging) and eases intubation. In addition, in certain embodiments, the recessed portion may change the outer wall diameter of the tube wall without affecting the inner wall diameter so that the work of breathing for the patient is not increased.

### BACKGROUND

The present disclosure relates generally to medical devices and, more particularly, to tracheal tubes that include controlled-profile regions.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tracheal tube (e.g endotracheal, nasotracheal, or transtracheal device) may be used to control the flow of gases into the trachea of a patient. Often, a seal between the outside of the tube and the interior wall of the tracheal lumen is required, allowing for generation of positive intrathoracic pressure distal to the seal and prevention of ingress of solid or liquid matter into the lungs from proximal to the seal.

Insertion of a tracheal tube involves the assistance of skilled medical personnel. In particular, doctors may wish to select a tracheal tube with a sufficiently large diameter to allow the tube to be easily inserted into the patient while providing the largest possible airway path for respiratory gases. For example, a tracheal tube with too small a tube diameter may be associated with an increased work of breathing for the patient. Conversely, a tracheal tube with too large a tube diameter presents certain disadvantages. For example, if the outer diameter of the tracheal tube is too large, it can become difficult to intubate through the larynx and trachea. In addition, the pressure of the tube against the tissue may result in discomfort for the patient. Accordingly, clinicians attempt to balance concerns about ease of intubation with concerns about providing a larger diameter for airflow when selecting a tracheal tube.

### BRIEF DESCRIPTION OF THE DRAWINGS

Advantages of the disclosed techniques may become apparent upon reading the following detailed description and upon reference to the drawings in which:

FIG. 1 is a perspective view of a tracheal tube with a controlled-profile cuff in accordance with embodiments of the present disclosure;

FIG. 2 is a perspective view of the tracheal tube of FIG. 1 with a deflated balloon cuff;

FIG. 3 is a side view of an exemplary tracheal tube with a controlled-profile cuff in accordance with embodiments of the present disclosure;

FIG. 4 is cross-sectional view of an exemplary tracheal tube with a controlled-profile cuff in accordance with embodiments of the present disclosure;

FIG. 5 is cross-sectional view of an alternative embodiment of a tracheal tube that includes multiple recessed portions to control a profile of a cuff;

FIG. 6 is a flow diagram of a manufacturing process for a tracheal tube with a controlled-profile cuff in accordance with embodiments of the present disclosure; and

FIG. 7 is a cross-sectional view of an assembly of an exemplary tracheal tube with a controlled-profile cuff in accordance with embodiments of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

During intubation, a tracheal tube is inserted into a patient's airway to isolate the lower airway and facilitate transfer of gases to and from the patient's lung. Certain airway products, such as endotracheal tubes and endobronchial tubes, are inserted through the patient's mouth and past upper respiratory anatomical features, e.g., the vocal cords. Other types of airway devices, such as tracheostomy tubes, may be inserted via a surgical incision to access the airway, i.e., a stoma. Regardless of the method of insertion, it is desirable for the outer diameter of the inserted tube to be sufficiently small to slide into the airway without damaging the device itself or causing undue discomfort for the patient. For airway devices that include inflatable balloon cuffs to seal the tracheal space, the profile of the cuff against the tube contributes to the overall outer diameter of the tracheal tube. A cuff is typically in a deflated state during insertion of a tracheal tube and is subsequently inflated after the tracheal tube is in place. However, even if deflated, the relatively thin cuff material tends to fold and wrinkle against the tube, particularly because the cuffs may be oversized relative to the trachea, which results in an excess of cuff material that folds against the tube and does not lie flat against the tubular body. Further, because the cuffs are relatively thin, the protruding cuff material may be damaged by snags on anatomical features, such as the teeth (e.g., in an endotracheal tube intubation) or the tissue of the stoma site (e.g., during percutaneous tracheostomy), which may increase the difficulty of inserting the tracheal tube. In the case of tracheostomy tube, to accommodate the size of the cuff around the tube, a surgeon may increase the size of the typical incision, which may increase the patient's healing time.

As described in detail below, embodiments of tracheal tubes having a controlled or reduced profile are provided herein. In particular, the profile of a balloon cuff associated with the tracheal tube is minimized to control the overall profile of the tracheal tube. The tracheal tubes as provided may facilitate intubation by reducing the overall outer diameter of the inserted device without sacrificing inner diameter space for ventilation. Further, the controlled-profile tracheal tubes may allow surgeons to employ relatively smaller incisions for tracheotomy tube insertion, which is advantageous because it is desirable to use the smallest possible incision site to promote patient recovery after removal of the tube. Further, because the smaller stoma size may more closely correlate to the outer diameter of an inserted tracheostomy tube, the tracheostomy tube may tend to remain in place relative to the stoma, which may improve patient comfort.

The tracheal tubes disclosed herein may include one or more recessed portions that accommodate an inflatable balloon cuff. The inflatable balloon cuff may be affixed to or around the recessed portion such that the protrusion of the cuff from the outer diameter of the tube is reduced or eliminated. In certain embodiments, the recessed portions allow the affixed portions of the cuff (i.e., the cuff shoulders), to be substantially flush with the outer diameter of the tube wall. In other embodiments, the material of the cuff, while deflated, conforms to and folds against a recessed portion of the tube to create a tracheal tube in which the overall protrusion of the cuff is reduced relative to the largest outer diameter of the tube. The present techniques are compatible with tracheal tubes formed by any suitable method. However, in particular embodiments, the techniques disclosed herein may be used in conjunction with extruded tracheal tubes. The present embodiments provide more cost-effective and less complex manufacturing solutions for reducing the profile of a tracheal tube.

Provided herein are controlled-profile tracheal tubes that may assume a reduced outer diameter, e.g., in and around the inflatable cuff, for improved insertion and removal. The tracheal tubes as provided herein are disposable rather than reusable, capable of providing differential mechanical ventilation to either or both lungs, and capable of supporting all other functions of standard endotracheal tubes (e.g. sealing, positive pressure generation, suctioning, irrigation, drug instillation, etc). The tracheal tubes can be used in conjunction with all acceptable auxiliary airway devices such as (e.g. heat and humidity conservers, mechanical ventilators, humidifiers, closed suction systems, scavengers, capnometers, oxygen analyzers, mass spectrometers, PEEP/CPAP devices, etc).

Furthermore, although the embodiments of the present disclosure illustrated and described herein are discussed in the context of tracheal tubes such as endotracheal tubes, it should be noted that presently contemplated embodiments may include an assembly end or assembly portion associated with any of a variety of suitable airway devices. For example, an assembly end as provided herein may be associated with a single-lumen tube, tracheostomy tube, a double-lumen tube (e.g., a Broncho-Cath™ tube), a specialty tube, or any other airway device with a main ventilation lumen. Indeed, any device with a ventilation lumen designed for use in an airway of a patient may include features for controlling the profile of the tube and/or cuff. As used herein, the term “tracheal tube” may include an endotracheal tube, a tracheostomy tube, a double-lumen tube, a bronchoblocking tube, a specialty tube, or any other airway device. In addition, the features for controlling profile as provided may be incorporated into catheters or other inserted or implantable medical devices.

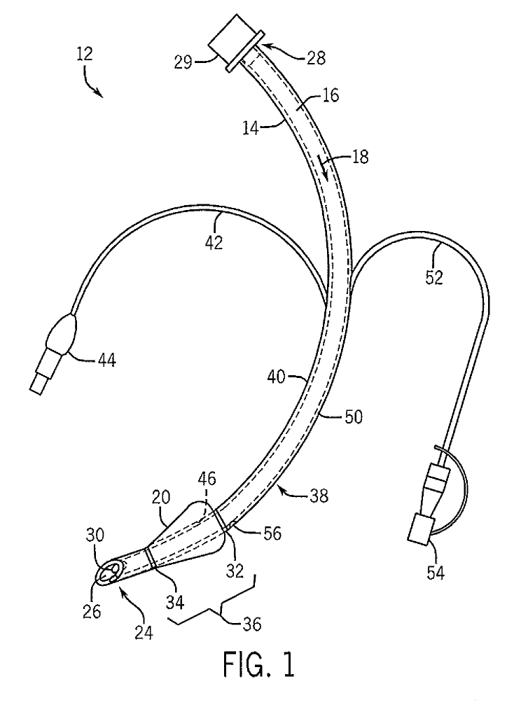

Turning now to the drawings, FIG. 1 is a perspective view of an exemplary tracheal tube 12 with features for controlling a profile of an inflatable cuff and configured to be placed in a patient's airway in accordance with aspects of the present disclosure. The tracheal tube 12 includes a central tubular body 14 that defines a ventilation lumen 16 that facilitates the transfer of gases to and from the lungs, e.g., as airflow into the lungs shown by arrow 18. The tracheal tube 12 includes an inflatable cuff 20 disposed towards a distal end 24. The distal end 24 terminates in an opening 26. A proximal end 28 of the tracheal tube 12 may connect to upstream airway devices (e.g., a ventilator) via connector 29. A Murphy eye 30 may be located on the tubular body 14 opposite the opening 26 to prevent airway occlusion when the tracheal tube 12 is improperly placed within the patient's trachea.

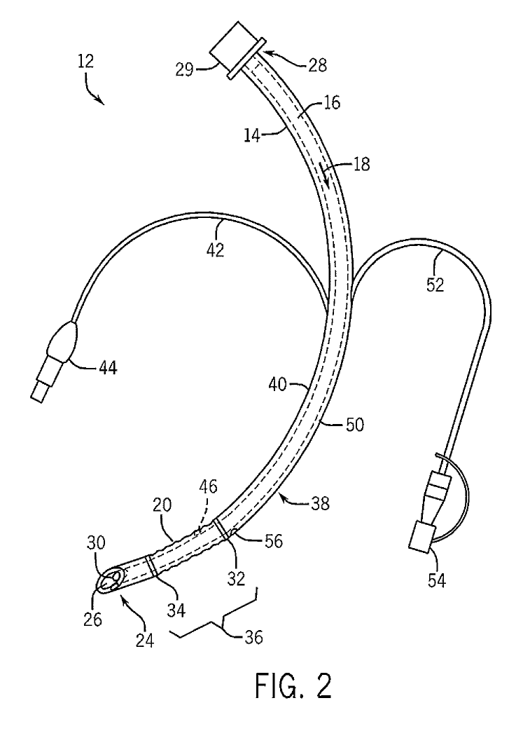

The cuff 20 is configured to seal the tracheal space once inflated against the tracheal walls. The cuff 20 is typically affixed to the tubular body 14 via a proximal shoulder 32 and a distal shoulder 34. As noted, the present disclosure relates to controlling the profile of the inflatable cuff 20. In certain embodiments, these features may be used in conjunction with oversized cuffs 20. In one embodiment, the inflatable cuff may be applied to a recessed portion 36 of the exterior surface 38 of the tubular body 14 that generally corresponds to the area covered by the cuff 20. That is, the recessed portion may include the portions of the tubular body 14 to which the proximal shoulder 32 and the distal shoulder 34 are attached. In this manner, the cuff 20, when deflated (see FIG. 2), conforms closely to the tubular body 14 without compromising the ability to seal in the inflated state, as shown in FIG. 2.

The cuff 20 may be inflated via inflation lumen 40 terminating at its proximal end in an inflation tube 42 connected to an inflation pilot balloon and valve assembly 44. The inflation lumen 40 terminates at its distal end in notch 46. Additionally, it should be noted that the cuff 20 may be any suitable cuff, such as a tapered cuff, a non-tapered cuff, and so forth. The tracheal tube 12 may also include a suction lumen 50 that extends from a location on the tracheal tube 12 positioned outside the body and that terminates in a suction tube 52 and suctioning port 54 for suctioning secretions through opening 56.

The tracheal tube 12 and the cuff 20 are formed from materials having suitable mechanical properties (such as puncture resistance, pin hole resistance, tensile strength), chemical properties (such as biocompatibility). In one embodiment, the walls of the cuff 20 are made of a polyurethane having suitable mechanical and chemical properties. An example of a suitable polyurethane is Dow Pellethane® 2363-80A. In another embodiment, the walls of the cuff 20 are made of a suitable polyvinyl chloride (PVC). In certain embodiments, the cuff 20 may be generally sized and shaped as a high volume, low pressure cuff that may be designed to be inflated to pressures between about 15 cm H₂O and 30 cm H₂O. However, it should be understood that the intracuff pressure may be dynamic. Accordingly, the initial inflation pressure of the cuff 20 may change over time or may change with changes in the seal quality or the position of the cuff 20 within the trachea. The tracheal tube 12 may be coupled to a respiratory circuit (not shown) that allows one-way flow of expired gases away from the patient and one-way flow of inspired gases towards the patient. The respiratory circuit, including the tracheal tube 12, may include standard medical tubing made from suitable materials such as polyurethane, polyvinyl chloride (PVC), polyethylene teraphthalate (PETP), low-density polyethylene (LDPE), polypropylene, silicone, neoprene, polytetrafluoroethylene (PTFE), or polyisoprene.

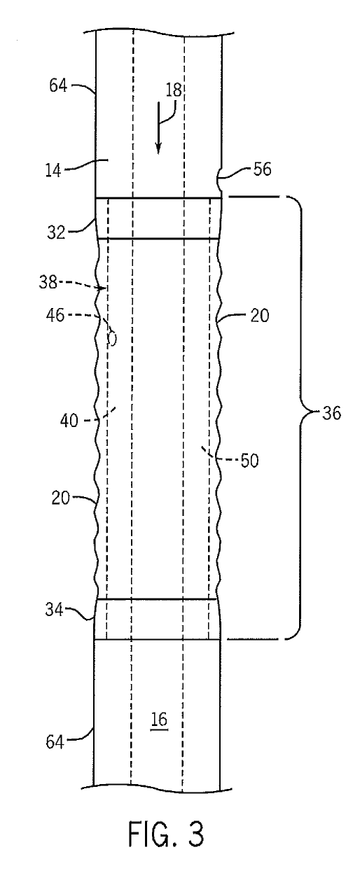

FIG. 3 is a partial side view of the cuff 20 in a deflated state. It should be noted that the controlled-profile features of the tracheal tube 12 are generally assumed when the cuff 20 is in the deflated state, e.g., during insertion or removal of the tracheal tube 12. During insertion and removal, the tracheal tube 12 and the associated cuff 20 pass through rigid anatomical features that may catch or snag the protruding cuff 20. In particular, to facilitate sealing of the trachea, the cuff 20 may an outer diameter portion that is larger than the estimated trachea size, e.g., 1.2 times-1.5 times larger. These portions may wrinkle or fold when inflated against the tracheal walls. These oversized portions, along with smaller diameter portions of the cuff 20, tend to form protrusions or wrinkles against the exterior surface 38 of the tubular body 14 when the cuff 20 is deflated. In the illustrated embodiment, the cuff 20 is positioned within the recessed portion 36 of the tubular body 14. Although the cuff 20 is generally wrinkled when deflated, the cuff 20 is sufficiently inset via recessed portion 36 that the overall protrusion of the cuff 20 relative to non-recessed portions 64 is reduced or no protrusion exists.

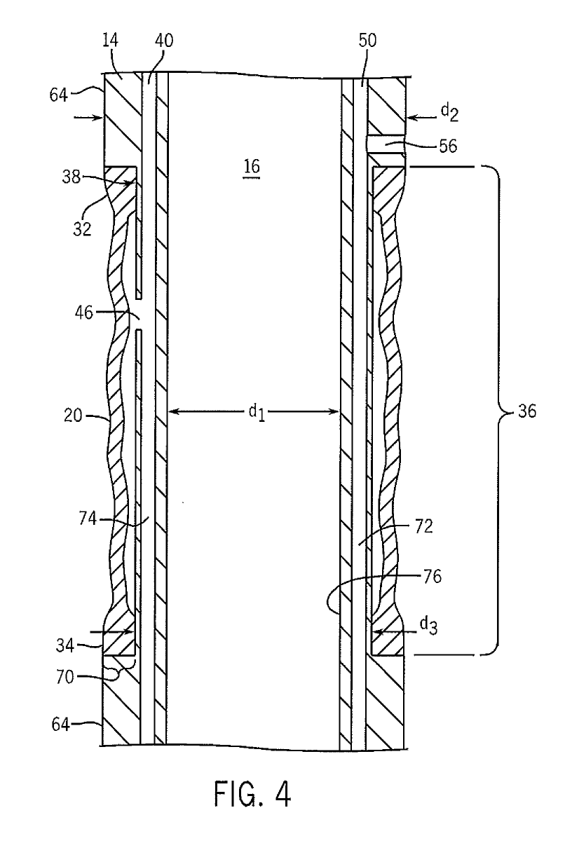

Further, this reduction in overall profile is accomplished with little to no change in the overall diameter of the ventilation lumen 16. As shown in cross section in FIG. 4, the inner diameter of lumen 16, shown as d₁, is substantially constant in the recessed portion 36 relative to the adjacent non-recessed portions 64. The recessed portion 36 may be formed by a reduction in wall thickness of the recessed portion 36 relative to non-recessed adjacent portions 64. As a result, the tubular body 14 may have a first, larger, outer diameter d₂ in the non-recessed portions 64 and a second, smaller, outer diameter d₃ in recessed portion 36 while maintaining the same inner diameter d₁. The reduction in wall thickness of the recessed portion 36 relative to the non-recessed portions 64 may be at least a cuff wall thickness 70 such that the cuff shoulders 32 and 34 are recessed or flush with the non-recessed portions 64. In certain embodiments, the recessed portion 36 may be recessed at least 0.1 mm, 0.5 mm, at least 1 mm, or at least 2 mm relative to the non-recessed portions 64. Further, the recessed portion 36 may be an annulus or a partial annulus about the tubular body 14.

In certain embodiments, the inflation lumen 40 and the suction lumen 50 are formed within the walls of the tubular body 14. Accordingly, the reduction in wall thickness may be limited by the positioning of these lumens. For suction lumen 50, because the suction opening 56 is above the cuff 20, the distal portion 72 of this lumen is generally unused and is plugged distal to the opening 56. For example, the plug may in the form of an inserted piece fitted within the distal portion 72 of the lumen 50 directly below the opening 56. In certain embodiments, the reduction in wall thickness may at least in part remove an unused distal portion 72 of the suction lumen 50 while still leaving the suctioning function of the lumen 50 intact. The cuff inflation lumen 40 terminates at opening 46. Similarly, the distal portion 74 of this lumen 40 distal to port 46 may be removed so long as the proximal cuff inflating portion is intact. Accordingly, the opening 46 may be positioned relative to the cuff 20 to allow a larger distal portion 74 to be removed. In other embodiments, for example in extruded assemblies, the lumens 40 and 50 may be formed relatively closer to the interior surface 76 of the tubular body 14 to allow a greater wall thickness reduction in the recessed portion 36. In other embodiments, the recessed portion 36 may take the form of a partial annulus in which the areas around the circumference of the tubular body 14 that correspond to the cuff inflation lumen 40 and the suction lumen 50 are not recessed. Such embodiments may allow control of the cuff profile while maintaining the integrity of the lumens 40 and 50.

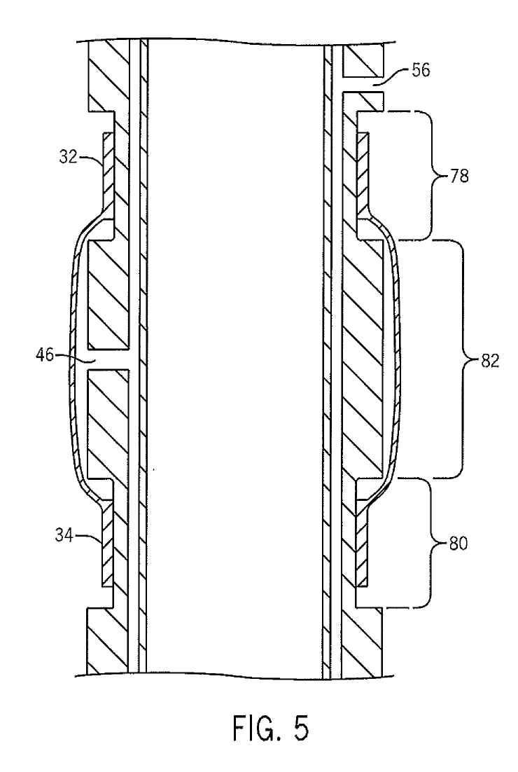

The recessed portion may encompass the entire cuff 20, or may be located in an area corresponding to only a portion of the cuff 20. Further, a tube 12 may include multiple recessed portions to control the profile of the cuff 20 or the tubular body 14. For example, FIG. 5 depicts a partial cross sectional view of an alternative embodiment including a first recessed portion 78 that corresponds to the location of the proximal cuff shoulder 32 and a second recessed portion 80 that corresponds to the location of the distal cuff shoulders 34. The recessed portions 78 and 80 are separated by either a non-recessed portion 82 or, in certain embodiments, a less recessed portion. In this manner, concerns about tube strength and resilience may be balanced with controlling the profile of the tracheal tube 12. For example, a discontinuous recess may result in a stronger tube. Further, a partially annular recess or a shaped or twisting recess (e.g. a spiral or corkscrew) may allow the folds of the deflated cuff to conform more closely to the tubular body 14 to reduce the overall profile of the tracheal tube 12 while maintaining sufficient wall strength.

The tracheal tubes 12 as provided herein may be formed by any suitable method. Further, the recess or reduction in wall thickness may be accomplished by any suitable process. For example, the tracheal tubes 12 may be formed via an extrusion process. Recessed portions (e.g., portion 36) of an extruded tube may be formed by a change in the parison thickness during extrusion. Alternatively, the extruded tube may be formed with walls of a generally constant thickness, and the recessed portions may be formed after the extrusion through additional processing steps. In one embodiment, a section of an extruded tube may be heated, stretched, and pushed in while in a formable state (e.g., while hot) to form the recessed portion. In other embodiments, the tracheal tube 12 may be a molded assembly that includes the appropriate recessed portions formed during the molding process.

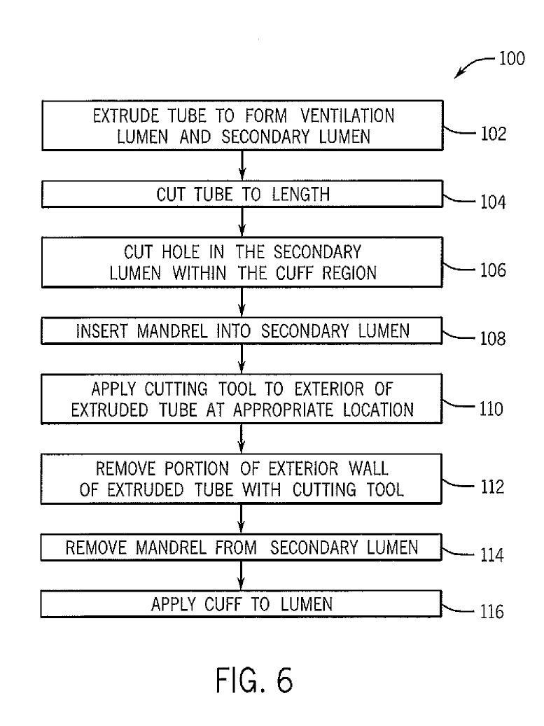

One embodiment of a method of forming a tracheal tube 12 as provided via a machining technique is illustrated by flow diagram 100 in FIG. 6. At step 102, a continuous tube or parison that forms a primary lumen (e.g., lumen 16) and one or more secondary lumens (e.g., cuff inflation lumen 40 and suction lumen 50) is formed. Alternatively, the tube may be a molded piece. The tube is cut to the appropriate length at step 104. A hole is cut into the secondary lumen within the cuff region at step 106. The tube is prepared for machining and a mandrel of the appropriate thickness (e.g. a suitable gauge wire) is inserted into the secondary lumen at the hole made in the previous step at step 108. The mandrel protects the secondary lumen from collapse during the forming as it heats up. At step 110, a cutting tool is applied to the exterior surface of the tube at the appropriate location along the tube, for example at a desired cuff application area, and at step 112, the cutting tool is used to machine off a designated thickness of the tube walls to form a recessed portion. After the recessed portion is formed, the mandrel is removed from the secondary lumen at step 114. The inflatable balloon cuff 20 may be affixed to the recessed portion at step 116 to form the tracheal tube 12.

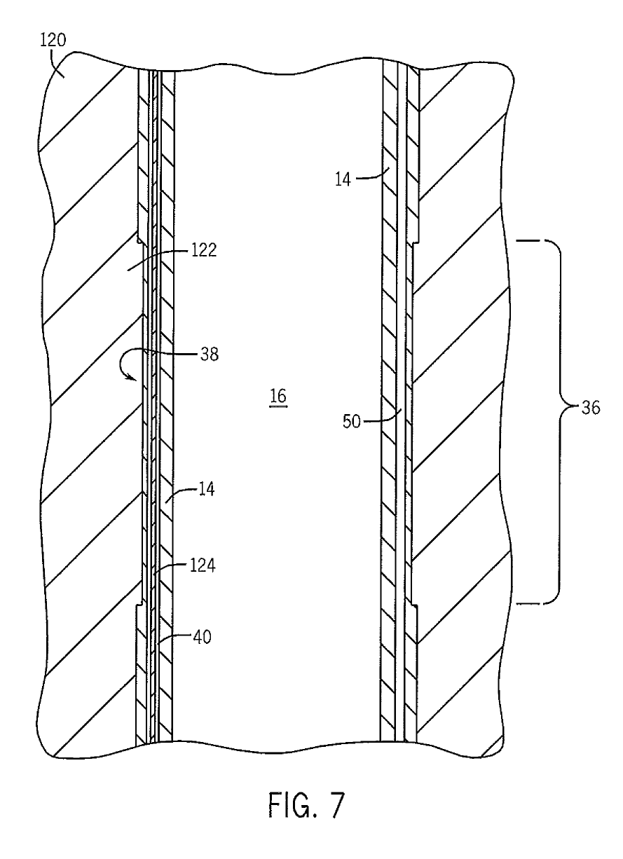

FIG. 7 is a cross-sectional view of a machining assembly for cutting a recessed portion. Cutting tool 120 is applied to the exterior surface 38 of the extruded section of the tube that will form the tubular body 14. The cutting tool 120 includes a protruding portion 122 that complements the size and shape of the desired recessed portion 36. As shown, the cutting tool 120 may substantially encircle the tubular body 14. In other embodiments, the cutting tool 120 may form a partial annulus and the tubular body may be spun while in contact with forming tool to form the recessed portion 36. As noted, a mandrel 124 may be inserted into any secondary lumens, such as cuff inflation lumen 40, to protect the lumen from collapse or splitting during the machining process. The mandrel 124 may be inserted from the top of the cut section or may be inserted via a hole or notch formed in the exterior surface 38.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A tracheal tube, comprising:
a conduit defining a passageway for delivering gas to a patient's lungs, the conduit comprising a peripheral recessed portion radially inward from an exterior surface of the conduit; and
an inflatable balloon cuff comprising a proximal shoulder region and a distal shoulder region affixed to the recessed portion of the conduit.

2. The tracheal tube of claim 1, wherein the recessed portion comprises a circumferential ring.

3. The tracheal tube of claim 1, wherein the proximal shoulder region and the distal region do not protrude from the exterior surface of the conduit.

4. The tracheal tube of claim 1, wherein the recessed portion has a substantially constant depth measured from the exterior surface.

5. The tracheal tube of claim 1, wherein the passageway has a substantially constant inner diameter along a length of the conduit.

6. The tracheal tube of claim 1, wherein the recessed portion is recessed at a depth approximately equal to a wall thickness of the inflatable balloon cuff.

7. The tracheal tube of claim 1, comprising a cuff inflation lumen, wherein the cuff inflation lumen comprises an opening in the recessed portion.

8. A tracheal tube, comprising:
a conduit defining a passageway for delivering gas to a patient's lungs, the conduit comprising an exterior surface having a recess; and
an inflatable balloon cuff comprising a proximal shoulder region and a distal shoulder region configured to be affixed to the exterior surface of the conduit at a first location and a second location, respectively, the first and second locations being situated within the recess.

9. The tracheal tube of claim 8, wherein the recess encompasses the first location and the second location and an area of the exterior surface between them.

10. The tracheal tube of claim 8, wherein the recess is sufficiently deep such that the proximal shoulder region is flush with a portion of the conduit proximal to the first location.

11. The tracheal tube of claim 8, wherein the recess is sufficiently deep such that the distal shoulder region is flush with a portion of the conduit distal to the first location.

12. The tracheal tube of claim 8, wherein the recess corresponds to the first location and comprising a second recess corresponding to the second location.

13. The tracheal tube of claim 8, wherein the recess is less than about 1 mm in thickness.

14. The tracheal tube of claim 8, wherein the recess is less than about 0.1 mm in thickness.

15. The tracheal tube of claim 8, wherein the recess comprises a circumferential ring.

16. The tracheal tube of claim 8, wherein the tracheal tube is configured to be coupled to at least one of a ventilator, a bag for ventilation, inspiration valving, expiration valving, or an air supply.

17. The tracheal tube of claim 8, wherein the tracheal tube comprises an endotracheal tube.

18. A method of manufacturing a tracheal tube, comprising:
affixing an inflatable balloon cuff to a recessed portion in an outer wall of an extruded tube, the outer wall being thinner in the recessed portion.

19. The method of claim 18, wherein the recessed portion comprises a circumferential ring.

20. The method of claim 18, wherein the recessed portion is formed by removing a section of the outer wall to create the recessed portion.

21. The method of claim 18, wherein the recessed portion is formed by stretching the extruded tube to create the recessed portion.
